(19) United States
(12) Patent Application Publication
(10) Pub. No.: US 2011/0275670 A1
(43) Pub. Date: Nov. 10, 2011

(54) New Compounds VII

(76) Inventors: Emma Chapman, Cambridge (GB); Michael Higginbottom, Cambridge (GB); Anne Viet-Anh Horgan (nee Nguyen), London (GB); Iain Simpson, Cambridge (GB)

(21) Appl. No.: 12/996,618

(22) PCT Filed: Jun. 4, 2009
(86) PCT No.: PCT/EP09/56892

§ 371 Date: Jul. 21, 2011

Related U.S. Application Data
(60) Provisional application No. 61/082,327, filed on Jul. 21, 2008.

(30) Foreign Application Priority Data
Jun. 4, 2008 (SE) 0801320-3

(51) Int. Cl. A61K 31/445; C07D 211/60; A61P 3/04; A61P 3/10; A61P 9/10; A61P 9/12; A61P 15/08; A61P 17/00; A61P 37/00; A61P 29/00; C07D 211/32; A61P 3/00

(52) U.S. Cl. 514/319; 546/233; 514/331; 546/234; 546/205; 546/230

(57) ABSTRACT

The present invention relates to new compounds of formula (I), to pharmaceutical compositions comprising these compounds and to the use of these compounds as leptin receptor modulator mimetics in the preparation of medicaments against conditions associated with weight gain, type 2 diabetes and dyslipidemias.

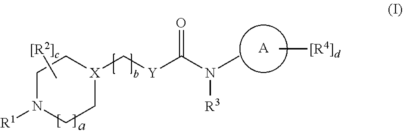

### FIELD OF THE INVENTION

The present application relates to new carbamate derivatives, to pharmaceutical compositions comprising these compounds and to the use of these compounds as leptin receptor modulator mimetics in the preparation of medicaments against conditions associated with weight gain, type 2 diabetes and dyslipidemias.

### BACKGROUND ART

The prevalence of obesity is increasing in the industrialized world. Typically, the first line of treatment is to offer diet and life style advice to patients, such as reducing the fat content of their diet and increasing their physical activity. However, some patients may also need to undergo drug therapy to maintain the beneficial results obtained from adapting the aforementioned diet and lifestyle changes.

Leptin is a hormone synthesized in fat cells that is believed to act in the hypothalamus to reduce food intake and body weight (see, e.g., Bryson, J. M. (2000) Diabetes, Obesity and Metabolism 2: 83-89).

It has been shown that in obese humans the ratio of leptin in the cerebrospinal fluid to that of circulating leptin is decreased (Koistinen et al., (1998) Eur. J. Clin. Invest. 28: 894-897). This suggests that the capacity for leptin transport into the brain is deficient in the obese state. Indeed, in animal models of obesity (NZO mouse and Koletsky rat), defects in leptin transport have been shown to result in reduced brain leptin content (Kastin, A. J. (1999) Peptides 20: 1449-1453; Banks, W. A. et al., (2002) Brain Res. 950: 130-136). In studies involving dietary-induced obese rodents (a rodent model that is believed to more closely resemble human obesity, see, e.g., Van Heek et al. (1997) J. Clin. Invest. 99: 385-390), excess leptin administered peripherally was shown to be ineffective in reducing food intake and body weight, whereas leptin injected directly into the brain was effective in reducing food intake and body weight. It has also been shown that in obese humans with excess circulating leptin, the signaling system became desensitized to the continual stimulation of the leptin receptors (Mantzoros, C. S. (1999) Ann. Intern. Med. 130: 671-680). Amgen has conducted clinical trials with recombinant methionyl human leptin. The results from these trials were mixed, as even in the presence of high plasma concentrations of leptin weight loss was variable, and the average weight reduction in the cohort of patients tested relatively small (Obesity Strategic Perspective, Datamonitor, 2001).

Several attempts at finding active fragments have been reported in the literature since the discovery of the leptin gene coding sequence. An example is by Samson et al. (1996) Endocrinol. 137: 5182-5185 which describes an active fragment at the N-terminal (22 to 56). This sequence was shown to reduce food intake when injected ICV whereas a sequence taken at the C-terminal was shown not to have any effect. Leptin fragments are also disclosed in International Patent Application WO 97/46585.

Other reports looking at the C-terminus part of the sequence reported a possible stimulation of luteinising hormone production by a 116-130 fragment (Gonzalez et al., (1999) Neuroendocrinology 70:213-220) and an effect on GH production following GHRH administration (fragment 126-140) (Hanew (2003) Eur. J. Endocrin. 149: 407-412).

Leptin has recently been associated with inflammation. It has been reported that circulating leptin levels rise during bacterial infection and in inflammation (see Otero, M et al. (2005) FEBS Lett. 579: 295-301 and references therein). Leptin can also act to increase inflammation by enhancing the release of pro-inflammatory cytokines TNF and IL-6 from inflammatory cells (Zarkesh-Esfahani, H. et al. (2001) J. Immunol. 167: 4593-4599). These agents in turn can contribute to the insulin resistance commonly seen in obese patients by reducing the efficacy of insulin receptor signaling (Lyon, C. J. et al. (2003) Endocrinol. 44: 2195-2200). Continuous low grade inflammation is believed to be associated with obesity (in the presence and absence of insulin resistance and Type II diabetes) (Browning et al. (2004) Metabolism 53: 899-903, Inflammatory markers elevated in blood of obese women; Mangge et al. (2004) Exp. Clin. Endocrinol. Diabetes 112: 378-382, Juvenile obesity correlates with serum inflammatory marker C-reactive protein; Maachi et al. (2004) Int. J. Obes. Relat. Metab. Disord. 28: 993-997, Systemic low grade inflammation in obese people). Leptin has also been implicated in the process of atherogenesis, by promoting lipid uptake into macrophages and endothelial dysfunction, thus promoting the formation of atherosclerotic plaques (see Lyon, C. J. et al. (2003) Endocrinol. 144: 2195-2200).

Leptin has also been shown to promote the formation of new blood vessels (angiogenesis) a process implicated in the growth of adipose tissue (Bouloumie A, et al. (1998) Circ. Res. 83: 1059-1066). Angiogenesis has also been implicated in diabetic retinopathy (Suganami, E. et al. (2004) Diabetes. 53: 2443-2448).

Angiogenesis is also believed to be involved with the growth of new blood vessels that feed abnormal tumour cells. Elevated leptin levels have been associated with a number of cancers, in particular breast, prostate and gastrointestinal cancers in humans (Somasundar P. et al. (2004) J. Surg. Res. 116: 337-349).

Leptin receptor agonists may also be used in the manufacture of a medicament to promote wound healing (Gorden, P. and Gavrilova, O. (2003) Current Opinion in Pharmacology 3: 655-659).

Further, it has been shown that elevating leptin signaling in the brain may represent an approach for the treatment of depressive disorders (Lu, Xin-Yun et al. (2006) PNAS 103: 1593-1598).

### DISCLOSURE OF THE INVENTION

It has surprisingly been found that compounds of formula (I) are effective in reducing body weight and food intake in rodents. While not wishing to be bound by theory, it is proposed that the compounds of formula I modulate the leptin receptor signaling pathway.

In some embodiments, compounds with leptin receptor agonistic like properties can be useful for the treatment of disorders relating to leptin signaling, as well as conditions associated with weight gain, such as obesity. The inventors hypothesized that small molecule CNS penetrant leptin mimetics would be able to by-pass the limiting uptake system into the brain. Further, assuming that this situation mirrors the human obese condition, the inventors believe that a CNS-penetrant leptinoid with a relatively long duration of action would make an effective therapy for the obese state and its attendant complications, in particular (but not limited to) diabetes.

In other embodiments, compounds with leptin receptor antagonistic like properties could be useful for the treatment of inflammation, atherosclerosis, diabetic retinopathy and nephropathy.

In a first aspect, the disclosure relates to a compound of formula (I),

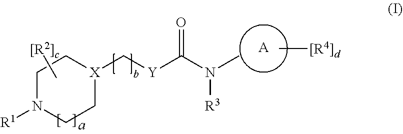

or a pharmaceutically acceptable salt, solvate, hydrate, geometrical isomer, tautomer, optical isomer or N-oxide thereof, wherein:
A is C₅₋₈-cycloalkyl;

### X is N or C(H);

### Y is O, N(R³) or CH₂;

R¹ is selected from hydrogen, C₁₋₆-alkyl, C₁₋₆-acyl (both optionally substituted with one or more substituents independently selected from halogen, hydroxy, cyano and C₁₋₆-alkoxy), phenyl and benzyl (both optionally substituted with one or more substituents independently is selected from halogen, hydroxy, cyano, nitro, CF₃, C₁₋₆-alkyl and C₁₋₆-alkoxy);
each R² is independently selected from halogen, hydroxy, C₁₋₆-alkyl and C₁₋₆-alkoxy (both optionally substituted with one or more substituents independently selected from halogen, hydroxy and C₁₋₆-alkoxy);
each R³ is independently selected from H and C₁₋₄-alkyl;
each R⁴ is independently selected from halogen, hydroxy, cyano, C₁₋₆-alkyl and C₁₋₆-alkoxy (both optionally substituted with one or more substituents independently selected from halogen, hydroxy and C₁₋₆-alkoxy), phenyl and benzyl (both optionally substituted with one or more substituents independently selected from halogen, hydroxy, cyano, nitro, CF₃, C₁₋₆-alkyl and C₁₋₆-alkoxy);
a is 0, 1 or 2;
b is 1 or 2; and
c and d are each independently 0, 1, 2 or 3;
provided that the compound is not selected from the group consisting of:
1-(3-chloro-2,2-dimethyl-1-oxopropyl)-N-[(cyclohexylamino)carbonyl]-4-piperidine-methanamine;
N-cyclohexyl-N′-[[1-[(4-methoxyphenyl)methyl]-4-piperidinyl]methyl]urea;
N-cyclohexyl-N,N′-dimethyl-N′-(4-piperidinylmethyl)urea;
4-piperidinylmethyl cyclohexylcarbamate;
2-[1-(phenylmethyl)-4-piperidinyl]ethyl cyclohexylcarbamate;
4-piperidinylmethyl cyclohexyl(methyl)carbamate;
2-(4-piperidinyl)ethyl cyclohexylcarbamate;
N-cyclopentyl-N,N′-dimethyl-N′-[2-(4-piperidinyl)ethyl]urea;
N′-cyclohexyl-N-methyl-N-(4-piperidinylmethyl)urea;
N-cyclohexyl-1-piperazinepropanamide;
N-(3-methylcyclohexyl)-1-piperazinepropanamide;
N-(2-methylcyclohexyl)-1-piperazinepropanamide;
N-(2,3-dimethylcyclohexyl)-1-piperazinepropanamide;
N-cyclohexyl-N′-[2-(1-piperazinyl)ethyl]urea;
2-(4-methylpiperazin-1-yl)ethyl (2-phenylbicyclo[2.2.1]hept-2-yl)carbamate;
N-cyclohexyl-1-piperazinebutanamide;
N-cyclohexyl-3-(1,4-diazepan-1-yl)propanamide;
3-(1,4-diazepan-1-yl)-N-(4-methylcyclohexyl)propanamide;
4-(4-chlorophenyl)-N-cyclohexyl-1-piperazinepropanamide;
4-(2-chlorophenyl)-N-cyclohexyl-N-methyl-1-piperazinepropanamide;
4-(3-chlorophenyl)-N-cyclohexyl-N-methyl-1-piperazinepropanamide;
4-(4-chlorophenyl)-N-cyclohexyl-N-methyl-1-piperazinepropanamide;
N-[2-[4-(2-methoxyphenyl)-1-piperazinyl]ethyl]-N′-(1-methylcyclohexyl)-urea;
N-cyclohexyl-4-(4-fluorophenyl)-N-methyl-1-piperazinepropanamide;
4-(2-methoxyphenyl)-N-(1-methylcyclohexyl)-1-piperazinepropanamide;
N-cyclohexyl-4-(4-methylphenyl)-1-piperazinepropanamide;
N-cyclohexyl-4-(4-methoxyphenyl)-N-methyl-1-piperazinepropanamide;
N-cyclohexyl-N-methyl-4-(4-methylphenyl)-1-piperazinepropanamide;
N-cyclohexyl-4-(2-methoxyphenyl)-N-methyl-1-piperazinepropanamide;
N-cyclohexyl-N-methyl-4-[3-(trifluoromethyl)phenyl]-1-piperazinepropanamide;
N-cyclopentyl-1-piperazinepropanamide;
N-cycloheptyl-1-piperazinepropanamide; and
N-cyclohexyl-N′-(4-piperidinylmethyl)urea.

In a preferred embodiment of the disclosure, X is C(H).

In another preferred embodiment, Y is O.

R¹ is preferably hydrogen, C₁₋₂-alkyl, C₁₋₂-alkoxy-C₁₋₂-alkyl, cyano-C₁₋₂-alkyl or benzyl, and more preferably methyl, 2-methoxyethyl, cyanomethyl or benzyl.

A is preferably a cyclopentane or bicyclo[2.2.1]heptane ring.

a is preferably 1, b is preferably 1, c is preferably 0 and d is preferably 0 or 1.

Specific preferred compounds according to the disclosure are those selected from the group consisting of:
(1-methylpiperidin-4-yl)methyl cyclopentylcarbamate;
(1-methylpiperidin-4-yl)methyl (1-phenylcyclopentyl)carbamate;
(1-methylpiperidin-4-yl)methyl bicyclo[2.2.1]hept-2-ylcarbamate;
piperidin-4-ylmethyl cyclopentylcarbamate;
[1-(2-methoxyethyl)piperidin-4-yl]methyl cyclopentylcarbamate;
(1-benzylpiperidin-4-yl)methyl cyclopentylcarbamate;
[1-(cyanomethyl)piperidin-4-yl]methyl cyclopentylcarbamate; and
[1-(2-methoxyethyl)piperidin-4-yl]methyl bicyclo[2.2.1]hept-2-ylcarbamate.

Another aspect of the present disclosure is a compound of formula (I) for use in therapy.

In a further aspect, the invention relates to a compound of formula (I) for use in the treatment or prevention of any of the disorders or conditions described herein.

In a yet further aspect, the invention relates to the use of the compounds of formula (I) in the manufacture of a medicament for the treatment or prevention of any of the disorders or conditions described herein.

In some embodiments, said compounds may be used for the treatment or prevention of a condition that is prevented, treated, or ameliorated by selective action via the leptin receptor.

In some embodiments, compounds of formula (I) may be used for the treatment or prevention of conditions (in particular, metabolic conditions) that are associated with weight gain. Conditions associated with weight gain include diseases, disorders, or other conditions that have an increased incidence in obese or overweight subjects. Examples include: lipodystrophy, HIV lipodystrophy, diabetes (type 2), insulin resistance, metabolic syndrome, hyperglycemia, hyperinsulinemia, dyslipidemia, hepatic steatosis, hyperphagia, hypertension, hypertriglyceridemia, infertility, a skin disorder associated with weight gain, macular degeneration. In some embodiments, compounds of the invention may also be used in the manufacture of a medicament for maintaining weight loss of a subject.

In some embodiments, compounds of formula (I) which are leptin receptor agonist mimetics may also be used to promote wound healing.

In some embodiments, compounds of formula (I) which are leptin receptor agonist mimetics may also be used for the treatment or prevention of conditions that cause a decrease in circulating leptin concentrations, and the consequent malfunction of the immune and reproductive systems. Examples of such conditions and malfunctions include severe weight loss, dysmenorrhea, amenorrhea, female infertility, immunodeficiency and conditions associated with low testosterone levels.

In some embodiments, compounds of formula (I) which are leptin receptor agonist mimetics may also be used for the treatment or prevention of conditions caused as a result of leptin deficiency, or a leptin or leptin receptor mutation.

In some other embodiments, compounds of formula (I) which are leptin receptor antagonist mimetics may be used for the treatment or prevention of inflammatory conditions or diseases, low level inflammation associated with obesity and excess plasma leptin and in reducing other complications associated with obesity including atherosclerosis, and for the correction of insulin resistance seen in Metabolic Syndrome and diabetes.

In some embodiments, compounds of formula (I) which are leptin receptor antagonist mimetics can be used for the treatment or prevention of inflammation caused by or associated with: cancer (such as leukemias, lymphomas, carcinomas, colon cancer, breast cancer, lung cancer, pancreatic cancer, hepatocellular carcinoma, kidney cancer, melanoma, hepatic, lung, breast, and prostate metastases, etc.); auto-immune disease (such as organ transplant rejection, lupus erythematosus, graft v. host rejection, allograft rejections, multiple sclerosis, rheumatoid arthritis, type I diabetes mellitus including the destruction of pancreatic islets leading to diabetes and the inflammatory consequences of diabetes); autoimmune damage (including multiple sclerosis, Guillam Barre Syndrome, myasthenia gravis); cardiovascular conditions associated with poor tissue perfusion and inflammation (such as atheromas, atherosclerosis, stroke, ischaemia-reperfusion injury, claudication, spinal cord injury, congestive heart failure, vasculitis, haemorrhagic shock, vasospasm following subarachnoid haemorrhage, vasospasm following cerebrovascular accident, pleuritis, pericarditis, the cardiovascular complications of diabetes); ischaemia-reperfusion injury, ischaemia and associated inflammation, restenosis following angioplasty and inflammatory aneurysms; epilepsy, neurodegeneration (including Alzheimer's Disease), arthritis (such as rheumatoid arthritis, osteoarthritis, rheumatoid spondylitis, gouty arthritis), fibrosis (for example of the lung, skin and liver), multiple sclerosis, sepsis, septic shock, encephalitis, infectious arthritis, Jarisch-Herxheimer reaction, shingles, toxic shock, cerebral malaria, Lyme's disease, endotoxic shock, gram negative shock, haemorrhagic shock, hepatitis (arising both from tissue damage or viral infection), deep vein thrombosis, gout; conditions associated with breathing difficulties (e.g. chronic obstructive pulmonary disease, impeded and obstructed airways, bronchoconstriction, pulmonary vasoconstriction, impeded respiration, chronic pulmonary inflammatory disease, silicosis, pulmonary sarcosis, cystic fibrosis, pulmonary hypertension, pulmonary vasoconstriction, emphysema, bronchial allergy and/or inflammation, asthma, hay fever, rhinitis, vernal conjunctivitis and adult respiratory distress syndrome); conditions associated with inflammation of the skin (including psoriasis, eczema, ulcers, contact dermatitis); conditions associated with inflammation of the bowel (including Crohn's disease, ulcerative colitis and pyresis, irritable bowel syndrome, inflammatory bowel disease); HIV (particularly HIV infection), cerebral malaria, bacterial meningitis, osteoporosis and other bone resorption diseases, osteoarthritis, infertility from endometriosis, fever and myalgia due to infection, and other conditions mediated by excessive anti-inflammatory cell (including neutrophil, eosinophil, macrophage and T-cell) activity.

In some embodiments, compounds of formula (I) which are leptin receptor antagonists mimetics may be used for the treatment or prevention of macro or micro vascular complications of type 1 or 2 diabetes, retinopathy, nephropathy, autonomic neuropathy, or blood vessel damage caused by ischaemia or atherosclerosis.

In some embodiments, compounds of formula (I) which are leptin receptor antagonist mimetics may be used to inhibit angiogenesis. Compounds that inhibit angiogenesis may be used for the treatment or prevention of obesity or complications associated with obesity. Compounds that inhibit angiogenesis may be used for the treatment or prevention of complications associated with inflammation diabetic retinopathy, or tumour growth particularly in breast, prostate or gastrointestinal cancer.

In a further aspect, the disclosure relates to a method for the treatment or prevention of any of the disorders or conditions described herein, which includes administering to a subject (e.g., a subject in need thereof, e.g., a mammal) an effective amount of a compound of formula I.

Methods delineated herein include those wherein the subject is identified as in need of a particular stated treatment. Identifying a subject in need of such treatment can be in the judgment of a subject or a health care professional and can be subjective (e.g. opinion) or objective (e.g. measurable by a test or diagnostic method).

In other aspects, the methods herein include those further comprising monitoring subject response to the treatment administrations. Such monitoring may include periodic sampling of subject tissue, fluids, specimens, cells, proteins, chemical markers, genetic materials, etc. as markers or indicators of the treatment regimen. In other methods, the subject is prescreened or identified as in need of such treatment by assessment for a relevant marker or indicator of suitability for such treatment.

In one embodiment, the invention provides a method of monitoring treatment progress. The method includes the step of determining a level of diagnostic marker (Marker) (e.g., any target or cell type delineated herein modulated by a compound herein) or diagnostic measurement (e.g., screen, assay) in a subject suffering from or susceptible to a disorder or symptoms thereof delineated herein, in which the subject has been administered a therapeutic amount of a compound herein sufficient to treat the disease or symptoms thereof. The level of Marker determined in the method can be compared to known levels of Marker in either healthy normal controls or in other afflicted patients to establish the subject's disease status. In preferred embodiments, a second level of Marker in the subject is determined at a time point later than the determination of the first level, and the two levels are compared to monitor the course of disease or the efficacy of the therapy. In certain preferred embodiments, a pre-treatment level of Marker in the subject is determined prior to beginning treatment according to this invention; this pre-treatment level of Marker can then be compared to the level of Marker in the subject after the treatment commences, to determine the efficacy of the treatment.

In certain method embodiments, a level of Marker or Marker activity in a subject is is determined at least once. Comparison of Marker levels, e.g., to another measurement of Marker level obtained previously or subsequently from the same patient, another patient, or a normal subject, may be useful in determining whether therapy according to the disclosure is having the desired effect, and thereby permitting adjustment of dosage levels as appropriate. Determination of Marker levels may be performed using any suitable sampling/expression assay method known in the art or described herein. Preferably, a tissue or fluid sample is first removed from a subject. Examples of suitable samples include blood, urine, tissue, mouth or cheek cells, and hair samples containing roots. Other suitable samples would be known to the person skilled in the art. Determination of protein levels and/or mRNA levels (e.g., Marker levels) in the sample can be performed using any suitable technique known in the art, including, but not limited to, enzyme immunoassay, ELISA, radio labeling/assay techniques, blotting/chemiluminescence methods, real-time PCR, and the like.

In some embodiments, it may be advantageous if a compound of formula (I) is able to penetrate the central nervous system. In other embodiments, it may be advantageous if a compound of formula (I) is not able to penetrate the CNS. In general, it is expected that compounds that are leptin receptor agonist mimetics may be particularly useful for the treatment or prevention of obesity, insulin resistance, or diabetes (particularly glucose intolerance) if these compounds can penetrate the CNS. A person of ordinary skill in the art can readily determine whether a compound can penetrate the CNS. A suitable method that may be used is described in the Biological Methods section.

A leptin receptor response may be measured in any suitable way. In vitro, this may be done be measuring leptin receptor signaling. For example, phosphorylation of Akt, STAT3, STAT5, MAPK, shp2 or the leptin receptor in response to binding of leptin or a compound of the invention to the leptin receptor may be measured. The extent of phosphorylation of Akt, STAT3, STAT5, MAPK, shp2 or the leptin receptor may be determined for example to by Western blotting or by ELISA. Alternatively, a STAT reporter assay may be used, for example STAT driven luciferase expression. A cell line expressing the leptin receptor may be used for such assays. In vivo, leptin receptor response may be measured by determining the reduction in food intake and body weight after administration of leptin or a compound of formula (I).

The Biological Methods below describe assays and methods that can be used to determine whether a compound of formula (I) is a leptin receptor agonist mimetic or a leptin receptor antagonist mimetic.

A compound of formula (I) may be administered with or without other therapeutic agents. For example, where it is desired to reduce inflammation, a compound may be administered with an anti-inflammatory agent (for example, disease modifying anti-rheumatic drugs such as methotrexate, sulphasalazine and cytokine inactivating agents, steroids, NSAIDs, cannabinoids, tachykinin modulators, or bradykinin modulators). Where it is desired to provide an anti-tumour effect, a compound may be administered with a cytotoxic agent (for example, methotrexate, cyclophosphamide) or another anti-tumour drug.

Compounds of formula (I) may be radio labeled (for example with tritium or radioactive iodine) for in vitro or in vivo applications, such as receptor displacement studies or receptor imaging.

### Definitions

The following definitions shall apply throughout the specification and the appended claims.

Unless otherwise stated or indicated, the term “C₁₋₆-alkyl” denotes a straight or branched alkyl group having from 1 to 6 carbon atoms. Examples of said C₁₋₆-alkyl include methyl, ethyl, n-propyl, iso-propyl, n-butyl, iso-butyl, sec-butyl, t-butyl, and straight- and branched-chain pentyl and hexyl. For parts of the range “C₁₋₆-alkyl” all subgroups thereof are contemplated such as C₁₋₅-alkyl, C₁₋₄-alkyl, C₁₋₃-alkyl, C₁₋₂-alkyl, C₂₋₆-alkyl, C₂₋₅-alkyl, C₂₋₄-alkyl, C₂₋₃-alkyl, C₃₋₆-alkyl, C₄₋₅-alkyl, etc.

Unless otherwise stated or indicated, the term “C₁₋₆-acyl” denotes a carbonyl group that is attached through its carbon atom to a hydrogen atom (i.e., a formyl group) or to a straight or branched C₁₋₅-alkyl group, where alkyl is defined as above. Examples of said C₁₋₆-acyl include formyl, acetyl, propionyl, n-butyryl, 2-methylpropionyl and n-pentoyl. For parts of is the range “C₁₋₆-acyl” all subgroups thereof are contemplated such as C₁₋₅-acyl, C₁₋₄-acyl, C₁₋₃-acyl, C₁₋₂-acyl, C₂₋₆-acyl, C₂₋₅-acyl, C₂₋₄-acyl, C₂₋₃-acyl, C₃₋₆-acyl, C₄₋₅-acyl, etc. If a C₁₋₆-acyl group is optionally substituted with one or more substituents independently selected from halogen, hydroxy, cyano and C₁₋₆-alkoxy, said substituent can not be attached to the carbonyl carbon atom.

Unless otherwise stated or indicated, the term “C₁₋₆-alkoxy” denotes a straight or branched alkoxy group having from 1 to 6 carbon atoms. Examples of said C₁₋₆-alkoxy include methoxy, ethoxy, n-propoxy, iso-propoxy, n-butoxy, iso-butoxy, sec-butoxy, t-butoxy, and straight- and branched-chain pentoxy and hexoxy. For parts of the range “C₁₋₆-alkoxy” all subgroups thereof are contemplated such as C₁₋₅-alkoxy, C₁₋₄-alkoxy, C₁₋₃-alkoxy, C₁₋₂-alkoxy, C₂₋₆-alkoxy, C₂₋₅-alkoxy, C₂₋₄-alkoxy, C₂₋₃-alkoxy, C₃₋₆-alkoxy, C₄₋₅-alkoxy, etc.

Unless otherwise stated or indicated, the term “C₅₋₈-cycloalkyl” denotes a mono- or bicyclic saturated hydrocarbon ring system having 5 to 8 carbon atoms. Bicyclic ring systems can be either fused or bridged. In a bridged cycloalkyl ring system, two non-adjacent carbon atoms of a monocyclic ring are linked by an alkylene bridge of between one and three additional carbon atoms. Examples of said C₅₋₈-cycloalkyl include cyclopentyl, cyclohexyl, bicyclo[2.2.1]heptyl, bicyclo[2.2.2]octyl and bicyclo[3.2.1]octyl. For parts of the range “C₅₋₈-cycloalkyl” all subgroups thereof are contemplated such as C₅₋₇-cycloalkyl, C₅₋₆-cycloalkyl, C₆₋₈-cycloalkyl, C₆₋₇-cycloalkyl, etc.

“Halogen” refers to fluorine, chlorine, bromine or iodine.

“Hydroxy” refers to the —OH radical.

“Nitro” refers to the —NO₂ radical.

“Cyano” refers to the —CN radical.

“Optional” or “optionally” means that the subsequently described event or circumstance may but need not occur, and that the description includes instances where the event or circumstance occurs and instances in which it does not.

The term “mammal” includes organisms, which include mice, rats, cows, sheep, pigs, rabbits, goats, and horses, monkeys, dogs, cats, and preferably humans. The subject may be a human subject or a non human animal, particularly a domesticated animal, such as a dog. “Pharmaceutically acceptable” means being useful in preparing a pharmaceutical composition that is generally safe, non-toxic and neither biologically nor otherwise undesirable and includes being useful for veterinary use as well as human pharmaceutical use.

“Treatment” as used herein includes prophylaxis of the named disorder or condition, or amelioration or elimination of the disorder once it has been established.

“An effective amount” refers to an amount of a compound that confers a therapeutic effect (e.g., treats, controls, ameliorates, prevents, delays the onset of, or reduces the risk of developing a disease, disorder, or condition or symptoms thereof) on the treated subject. The therapeutic effect may be objective (i.e., measurable by some test or marker) or subjective (i.e., subject gives an indication of or feels an effect).

“Prodrugs” refers to compounds that may be converted under physiological conditions or by solvolysis to a biologically active compound of formula (I). A prodrug may be inactive when administered to a subject in need thereof, but is converted in vivo to an active compound of formula (I). Prodrugs are typically rapidly transformed in vivo to yield the parent compound, e.g. by hydrolysis in the blood. The prodrug compound usually offers advantages of solubility, tissue compatibility or delayed release in a mammalian organism (see Silverman, R. B., The Organic Chemistry of Drug Design and Drug Action, 2^{nd} Ed., Elsevier Academic Press (2004), pp. 498-549). Prodrugs may be prepared by modifying functional groups, such as a hydroxy, amino or mercapto groups, present in a compound of formula (I) in such a way that the modifications are cleaved, either in routine manipulation or in vivo, to the parent compound. Examples of prodrugs include, but are not limited to, acetate, formate and succinate derivatives of hydroxy functional groups or phenyl carbamate derivatives of amino functional groups.

Throughout the specification and the appended claims, a given chemical formula or name shall also encompass all salts, hydrates, solvates, N-oxides and prodrug forms thereof. Further, a given chemical formula or name shall encompass all tautomeric and stereoisomeric forms thereof. Stereoisomers include enantiomers and diastereomers. Enantiomers can be present in their pure forms, or as racemic (equal) or unequal mixtures of two enantiomers. Diastereomers can be present in their pure forms, or as mixtures of diastereomers. Diastereomers also include geometrical isomers, which can be present in their pure cis or trans forms or as mixtures of those.

The compounds of formula (I) may be used as such or, where appropriate, as pharmacologically acceptable salts (acid or base addition salts) thereof. The is pharmacologically acceptable addition salts mentioned below are meant to comprise the therapeutically active non-toxic acid and base addition salt forms that the compounds are able to form. Compounds that have basic properties can be converted to their pharmaceutically acceptable acid addition salts by treating the base form with an appropriate acid. Exemplary acids include inorganic acids, such as hydrogen chloride, hydrogen bromide, hydrogen iodide, sulphuric acid, phosphoric acid; and organic acids such as formic acid, acetic acid, propanoic acid, hydroxyacetic acid, lactic acid, pyruvic acid, glycolic acid, maleic acid, malonic acid, oxalic acid, benzenesulphonic acid, toluenesulphonic acid, methanesulphonic acid, trifluoroacetic acid, fumaric acid, succinic acid, malic acid, tartaric acid, citric acid, salicylic acid, p-aminosalicylic acid, pamoic acid, benzoic acid, ascorbic acid and the like. Exemplary base addition salt forms are the sodium, potassium, calcium salts, and salts with pharmaceutically acceptable amines such as, for example, ammonia, alkylamines, benzathine, and amino acids, such as, e.g. arginine and lysine. The term addition salt as used herein also comprises solvates which the compounds and salts thereof are able to form, such as, for example, hydrates, alcoholates and the like.

### Compositions

For clinical use, the compounds of formula (I) are formulated into pharmaceutical formulations for various modes of administration. It will be appreciated that the compounds may be administered together with a physiologically acceptable carrier, excipient, or diluent. The pharmaceutical compositions may be administered by any suitable route, preferably by oral, rectal, nasal, topical (including buccal and sublingual), sublingual, transdermal, intrathecal, transmucosal or parenteral (including subcutaneous, intramuscular, intravenous and intradermal) administration.

Other formulations may conveniently be presented in unit dosage form, e.g., tablets and sustained release capsules, and in liposomes, and may be prepared by any methods well known in the art of pharmacy. Pharmaceutical formulations are usually prepared by mixing the active substance, or a pharmaceutically acceptable salt thereof, with conventional pharmaceutically acceptable carriers, diluents or excipients. Examples of excipients are is water, gelatin, gum arabicum, lactose, microcrystalline cellulose, starch, sodium starch glycolate, calcium hydrogen phosphate, magnesium stearate, talcum, colloidal silicon dioxide, and the like. Such formulations may also contain other pharmacologically active agents, and conventional additives, such as stabilizers, wetting agents, emulsifiers, flavouring agents, buffers, and the like. Usually, the amount of active compounds is between 0.1-95% by weight of the preparation, preferably between 0.2-20% by weight in preparations for parenteral use and more preferably between 1-50% by weight in preparations for oral administration.

The formulations can be further prepared by known methods such as granulation, compression, microencapsulation, spray coating, etc. The formulations may be prepared by conventional methods in the dosage form of tablets, capsules, granules, powders, syrups, suspensions, suppositories or injections. Liquid formulations may be prepared by dissolving or suspending the active substance in water or other suitable vehicles. Tablets and granules may be coated in a conventional manner. To maintain therapeutically effective plasma concentrations for extended periods of time, the compounds may be incorporated into slow release formulations.

The dose level and frequency of dosage of the specific compound will vary depending on a variety of factors including the potency of the specific compound employed, the metabolic stability and length of action of that compound, the patient's age, body weight, general health, sex, diet, mode and time of administration, rate of excretion, drug combination, the severity of the condition to be treated, and the patient undergoing therapy. The daily dosage may, for example, range from about 0.001 mg to about 100 mg per kilo of body weight, administered singly or multiply in doses, e.g. from about 0.01 mg to about 25 mg each. Normally, such a dosage is given orally but parenteral administration may also be chosen.

### Preparation of Compounds of the Invention

The compounds of formula (I) above may be prepared by, or in analogy with, conventional methods. Formation of the central urethane linker is the key synthetic step in preparing the compounds formula (I). A large number of activating reagents can be used for the formation of a urethane linker e.g. phosgene to form chloroformate of alcohols, or carbonyldiimidazole (CDI) to form imidazole carboxylates. Typically the urethane linkers incorporated into compounds of formula (I) have been synthesized utilizing bis-(4-nitrophenyl)carbonate or p-nitrophenyl chloroformate as the activating agent, or by condensation of an alcohol with an isocyanate intermediate. The preparation of intermediates and compounds according to the examples of the present invention may in particular be illuminated by the following Schemes 1 and 2. Definitions of variables in the structures in the schemes herein are commensurate with those of corresponding positions in the formulae delineated herein.

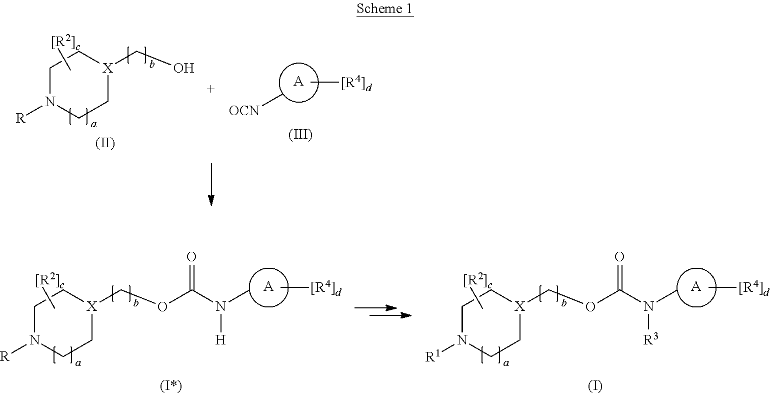

wherein R is R¹ or a protecting group; and
A, X, R¹-R⁴ and a-d are as defined in formula (I).

Compounds of formula (I) can easily be prepared in only a few steps. In one possible route, an alcohol of formula (II) is condensed with the appropriate isocyanate derivative of formula (III) in a suitable solvent (such as THF or toluene) to give a compound of formula (I*), which is either a compound of formula (I) or a suitably protected derivative thereof. If required, a compound of formula (I*) can subsequently be transformed into the desired compound of formula (I) in one or more additional steps.

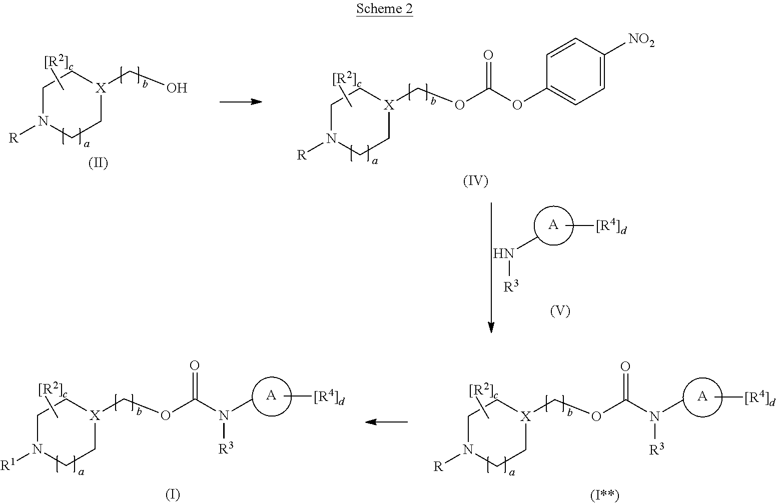

wherein R is R′ or a protecting group; and
A, X, R¹-R⁴ and a-d are as defined in formula (I).

Alternatively, an alcohol of formula (II) can be activated with bis-(4-nitrophenyl)carbonate or p-nitrophenyl chloroformate in the presence of a base (such as NMM) in an suitable solvent (such as DCM) to give the corresponding carbonate of formula (IV). The carbonate intermediate (IV) is then subsequently treated with the appropriate amine of formula (V) in the presence of a base (such as DIPEA) and optionally an activating agent (such as DMAP) in a suitable solvent (such as DMF) to give a compound of formula (I**), which is either a compound of formula (I) or a suitably protected derivative thereof. If required, a compound of formula (I**) can subsequently be transformed into the desired compound of formula (I).

The necessary starting materials for preparing the compounds of formula (I) are either commercially available, or may be prepared by methods known in the art.

The processes described below in the experimental section may be carried out to give a compound of the invention in the form of a free base or as an acid addition salt. A pharmaceutically acceptable acid addition salt may be obtained by dissolving the free base in a suitable organic solvent and treating the solution with an acid, in accordance with conventional procedures for preparing acid addition salts from base compounds. Examples of addition salt forming acids are mentioned above.

The compounds of formula (I) may possess one or more chiral carbon atoms, and they may therefore be obtained in the form of optical isomers, e.g., as a pure enantiomer, or as a mixture of enantiomers (racemate) or as a mixture containing diastereomers. The separation of mixtures of optical isomers to obtain pure enantiomers is well known in the art and may, for example, be achieved by fractional crystallization of salts with optically active (chiral) acids or by chromatographic separation on chiral columns.

The chemicals used in the synthetic routes delineated herein may include, for example, solvents, reagents, catalysts, and protecting group and deprotecting group reagents. Examples of protecting groups are t-butoxycarbonyl (Boc), benzyl and trityl (triphenylmethyl). The methods described above may also additionally include steps, either before or after the steps described specifically herein, to add or remove suitable protecting groups in order to ultimately allow synthesis of the compounds. In addition, various synthetic steps may be performed in an alternate sequence or order to give the desired compounds. Synthetic chemistry transformations and protecting group methodologies (protection and deprotection) useful in synthesizing applicable compounds are known in the art and include, for example, those described in R. Larock, Comprehensive Organic Transformations, VCH Publishers (1989); T. W. Greene and P. G. M. Wuts, Protective Groups in Organic Synthesis, 3^{rd} Ed., John Wiley and Sons (1999); L. Fieser and M. Fieser, Fieser and Fieser's Reagents for Organic Synthesis, John Wiley and Sons (1994); and L. Paquette, ed., Encyclopedia of Reagents for Organic Synthesis, John Wiley and Sons (1995) and subsequent editions thereof.

The following abbreviations have been used:

| | |
|---|---|
| aq | Aqueous |
| Boc | tert-Butoxy carbonyl |
| DCM | Dichloromethane |
| DIPEA | N,N-Diisopropylethylamine |
| DMAP | N,N-Dimethylaminopyridine |
| DMF | N,N-Dimethylformamide |
| ES⁺ | Electrospray |
| Et₂O | Diethyl ether |
| EtOAc | Ethyl acetate |
| HIV | Human immunodeficiency virus |
| HPLC | High performance liquid chromatography |
| ICV | Intracerebroventricular |
| LCMS | Liquid Chromatography Mass Spectrometry |
| M | Molar |
| [MH]⁺ | Protonated molecular ion |
| NEt₃ | Triethylamine |
| NMM | N-methyl morpholine |
| RP | Reverse Phase |
| sat | Saturated |
| tert | Tertiary |
| TFA | Trifluoroacetic acid |
| THF | Tetrahydrofuran |

Embodiments of the disclosure are described in the following examples with reference to the accompanying drawings, in which:

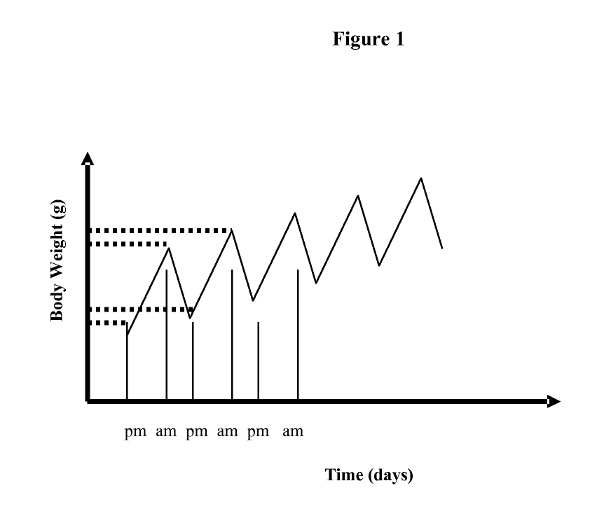

FIG. 1 is a schematic drawing illustrating weight gain and weight loss in mice during dark and light phases, respectively. The graph illustrates the large nocturnal weight increase versus the comparatively small body weight change over 24 hours

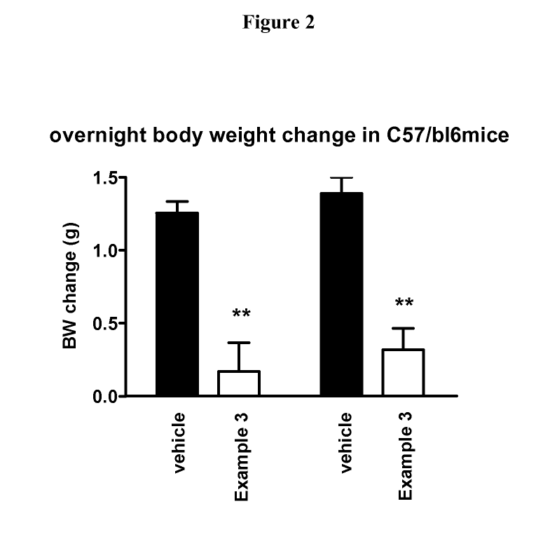

FIG. 2 shows the effect of Example 3 on the body weight in mice between the beginning of the dark phase and the beginning of the light phase (pm-am).

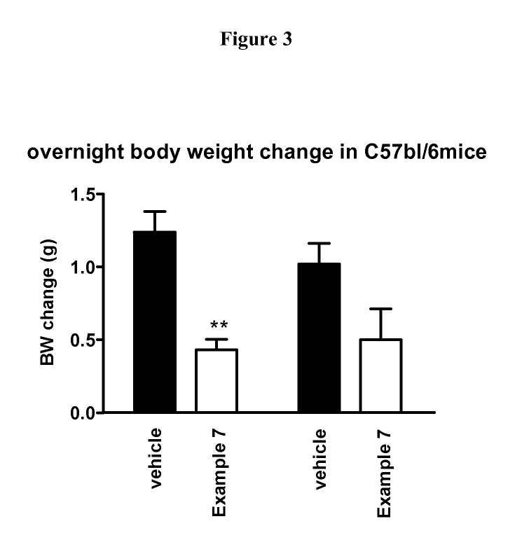

FIG. 3 shows the effect of Example 7 on the body weight in mice between the beginning of the dark phase and the beginning of the light phase (pm-am).

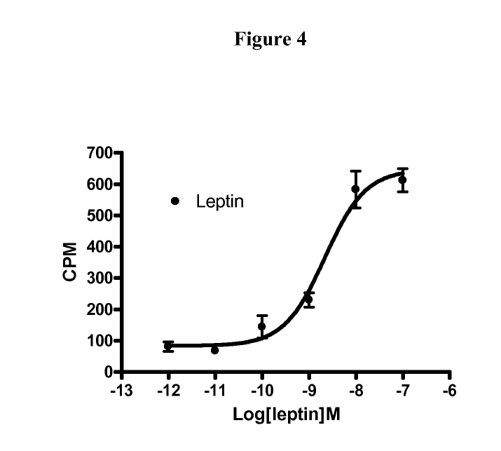

FIG. 4 shows the concentration-dependent increase in [³H]-thymidine incorporation by JEG-3 cells for leptin

The recitation of a listing of chemical groups in any definition of a variable herein includes definitions of that variable as any single group or combination of listed groups. The recitation of an embodiment herein includes that embodiment as any single embodiment or in combination with any other embodiments or portions thereof.

The disclosure will now be further illustrated by the following non-limiting examples. The specific examples below are to be construed as merely illustrative, and not limitative of the is remainder of the disclosure in any way whatsoever. Without further elaboration, it is believed that one skilled in the art can, based on the description herein, utilize the present disclosure to its fullest extent. All references and publications cited herein are hereby incorporated by reference in their entirety.

### EXAMPLES AND INTERMEDIATE COMPOUNDS

### Experimental Methods

All reagents were commercial grade and were used as received without further purification, unless otherwise specified. Commercially available anhydrous solvents were used for reactions conducted under inert atmosphere. Reagent grade solvents were used in all other cases, unless otherwise specified. Analytical LCMS was performed on a Waters ZQ mass spectrometer connected to an Agilent 1100 HPLC system. Analytical HPLC was performed on an Agilent 1100 system. High-resolution mass spectra (HRMS) were obtained on an Agilent MSD-TOF connected to an Agilent 1100 HPLC system. During the analyses the calibration was checked by two masses and automatically corrected when needed. Spectra are acquired in positive electrospray mode. The acquired mass range was m/z 100-1100. Profile detection of the mass peaks was used. Normal phase is chromatography was performed on a Flash Master Personal system equipped with Strata SI-1 silica gigatubes. Reverse phase chromatography was performed on a Gilson system equipped with Merck LiChoprep® RP-18 (40-63 μm) 460×26 mm column, 30 mL/min, gradient of methanol in water from 0% to 100%. Preparative HPLC was performed on a Gilson system equipped with Phenomenex Hydro RP 150×20 mm, 20 mL/min, gradient of acetonitrile in water from 0% to 100%. The compounds were automatically named using ACD 6.0.

Analytical HPLC and LCMS data were obtained with:

System A: Phenomenex Synergi Hydro RP (30×4.6 mm, 4 μm), gradient 5-100% CH₃CN in H₂O (+0.1% HCO₂H), 1.5 mL/min, gradient time 1.75 min, 200-300 nm, 30° C.; or
System B: Phenomenex Synergi Hydro RP (150×4.6 mm, 4 μm), gradient 5-100% CH₃CN (+0.085% TFA) in H₂O (+0.1% TFA), 1.5 mL/min, gradient time 7 min, 200-300 nm, 30° C.;
System C: Phenomenex Synergi Hydro RP (150×4.6 mm, 4 μm), gradient 5-100% CH₃CN in H₂O (+0.1% HCO₂H), 1.0 mL/min, gradient time 8 min, 200-300 nm, 30° C.;

### Intermediate 1

### tert-Butyl 4-(hydroxymethyl)piperidine-1-carboxylate

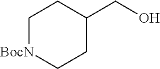

To a solution of 4-piperidine methanol (10.0 g, 86.8 mmol) and DIPEA (15 mL, 86.6 mmol) in DCM (200 mL) was added di-tert-butyl dicarbonate (18.95 g, 86.8 mmol) portion-wise. The reaction mixture was stirred at room temperature for 19 hours. The reaction mixture was washed with 2M aq HCl (150 mL) and 1M aq Na₂CO₃ solution (150 mL), dried (MgSO₄) and the solvent evaporated in vacuo to yield tert-butyl 4-(hydroxymethyl)piperidine-1-carboxylate (16.1 g, 87%) as a white solid.

Analytical LCMS: (System A, R_{T}=1.80 min), ES⁺: 216.3 [MH]⁺.

### Intermediate 2

### (1-Methylpiperidin-4-yl)methanol

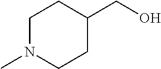

A solution of tert-butyl 4-(hydroxymethyl)piperidine-1-carboxylate (1.94 g, 9.0 mmol) in THF (15 mL) was added drop-wise to 1M solution of LiAlH₄ in THF (13.5 mL, 13.5 mmol) under argon. The reaction mixture was stirred at room temperature for 17 hours, cooled to 0° C. and quenched by the dropwise addition of a mixture of THF and water (1:1 ratio, 1.5 mL). 4M aq NaOH solution (0.6 mL), and water (2.0 mL) were added and the mixture stirred at room temperature for 2 hours. A white solid was removed by filtration. The filtrate was purified by using an Isolute HM-N cartridge, eluting with EtOAc (200 mL). The eluent was concentrated in vacuo yielding (1-methylpiperidin-4-yl)methanol (1.02 g, 88%) as a yellow oil.

Analytical LCMS: (System A, R_{T}=0.32 min), ES⁺: 130.3 [MH]⁺.

### Intermediate 3

### (1-(2-Methoxyethyl)piperidin-4-yl)methanol

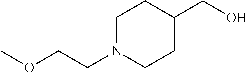

To a stirred solution of piperidin-4-yl-methanol (3.13 g, 27.2 mmol), DMAP (50 mg) and NEt₃ (7.0 mL, 50.6 mmol) in DCM (30 mL) at 0° C. was added methoxy-acetyl chloride (5.0 mL, 54.8 mmol) in 0.5 mL aliquots. The reaction mixture was stirred for 2 hours and then diluted with DCM (70 mL). The reaction mixture was washed with 1M aq HCl solution (100 mL), 1M aq Na₂CO₃ solution (100 mL), dried (MgSO₄) and concentrated in vacuo to give (1-(2-methoxyacetyl)piperidin-4-yl)methyl 2-methoxyacetate (6.5 g, 92%) as a yellow oil.

A solution of (1-(2-methoxyacetyl)piperidin-4-yl)methyl 2-methoxyacetate (6.5 g, 25.1 mmol) in THF (10 mL) was added dropwise to a stirred 1M solution of LiAlH₄ in THF (55.0 mL, 55.0 mmol) under argon. The reaction mixture was stirred at room temperature for 2 days, cooled to 0° C. and quenched by the drop wise addition of water (2.0 mL). 0.2M aq NaOH solution (2.0 mL) and water (5.0 mL) were added the mixture stirred at room temperature for 3 h. A white solid was removed by filtration. The filtrate was concentrated in vacuo and purified by using an Isolute HM-N cartridge, eluting with EtOAc. The eluent is was dried in vacuo to give (1-(2-methoxyethyl)piperidin-4-yl)methanol (3.65 mg, 84%) as a yellow oil.

Analytical LCMS: (System A, R_{T}=0.35 min), ES⁺: 174.2 [MH]⁺.

### Example 1

### (1-Methylpiperidin-4-yl)methyl cyclopentylcarbamate hydrochloride

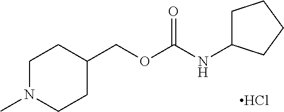

(1-Methylpiperidin-4-yl)methanol (Intermediate 2; 4.67 g, 35.7 mmol) was dissolved in anhydrous THF (40 mL) and cyclopentyl isocyanate (3.62 mL, 32.1 mmol) was added. After stirring at room temperature for 16 hours, the reaction mixture was concentrated in vacuo. The residue was dissolved in hot EtOAc (5 mL), cooled to 0° C., filtered and the filtrate concentrated in vacuo. A 60% portion of the crude product was purified by normal phase chromatography (gradient eluting with MeOH in DCM from 0% to 10% with 1% aq NH₃ in mobile phase). The residue was dissolved in acetonitrile (20 mL), 2M HCl in Et₂O (3 mL, 6 mmol) was added and the solution concentrated in vacuo. The residue was dissolved in 2M aq HCl (25 mL), washed with EtOAc (3×25 mL), filtered and dried in vacuo to give (1-methylpiperidin-4-yl)methyl cyclopentylcarbamate hydrochloride (1.32 g, 25%) as a hygroscopic white solid.

Analytical LCMS: purity 99.1% (System C, R_{T}=4.84 min), ES⁺: 241.3 [MH]⁺; HRMS calcd for C₁₃H₂₄N₂O₂: 240.1838, found 240.1843.

### Example 2

### (1-Methylpiperidin-4-yl)methyl (1-phenylcyclopentyl)carbamate formate

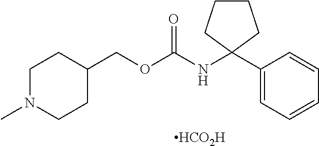

(1-Methylpiperidin-4-yl)methanol (Intermediate 2; 0.430 g, 2.0 mmol) and 1-phenylcyclopentyl isocyanate (0.445 g, 2.0 mmol; prepared according to the procedure described by Kaiser, C. and Weinstock J., Org. Synth. Coll., Vol. 7, 433) were dissolved in anhydrous toluene (5 mL) and heated under reflux for 2 h before removing the volatiles in vacuo. The residue was dissolved in MeOH (3 mL), concentrated in vacuo and then purified by reverse phase chromatography (gradient eluting with MeOH in water, with 1% is formic acid in each solvent, from 0% to 100%) to give (1-methylpiperidin-4-yl)methyl (1-phenylcyclopentyl)carbamate formate (110 mg, 15%) as a transparent oil.

Analytical LCMS: purity 98.9% (System C, R_{T}=5.93 min), ES⁺: 317.1 [MH]⁺; HRMS calcd for C₁₉H₂₈N₂O₂: 316.2151, found 316.2158.

### Example 3

### (1-Methylpiperidin-4-yl)methyl bicyclo[2.2.1]hept-2-ylcarbamate hydrochloride

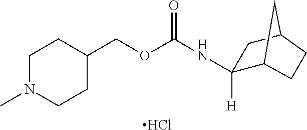

To a solution of bis-4-nitrophenylcarbonate (7.06 g, 23.2 mmol) in DCM (100 mL) was added a solution of (1-methylpiperidin-4-yl)methanol (Intermediate 2; 2.50 g, 19.3 mmol) in DCM (50 mL) followed by NMM (1.70 mL, 15.5 mmol). The reaction mixture was stirred for 90 hours, concentrated in vacuo, the residue dissolved in EtOAc (80 mL) and then washed with 1M aq Na₂CO₃ solution to remove p-nitrophenol. The organic layer was dried (MgSO₄) and evaporated in vacuo to give (1-methylpiperidin-4-yl)methyl 4-nitrophenyl carbonate (4.18 g, 73%) as a yellow solid.

Analytical LCMS: (System A, R_{T}=1.59 min), ES⁺: 295.1 [MH]⁺.

To a solution of (1-methylpiperidin-4-yl)methyl 4-nitrophenyl carbonate (587 mg, 2.0 mmol) in DMF (20 mL) was added DIPEA (0.696 mL, 2.0 mmol), DMAP (10 mg, catalytic) and exo-2-aminonorbornane (0.356 mL, 3.0 mmol). The reaction mixture was stirred at room temperature for 17 hours and then concentrated in vacuo. The residue was dissolved in 1M aq HCl solution (25 mL) and washed with DCM (25 mL) and EtOAc (2×25 mL). The combined organic phases were dried (MgSO₄), filtered, concentrated in vacuo and purified by reverse phase chromatography (gradient eluting with MeOH in water, with 1% formic acid in each solvent, from 0% to 100%). The colourless oil obtained was dissolved in MeOH (5 mL), 2M HCl in Et₂O added (0.5 mL, 1.0 mmol) and concentrated in vacuo to give (1-methylpiperidin-4-yl)methyl bicyclo[2.2.1]hept-2-ylcarbamate is hydrochloride (152 mg, 25%) as a white solid.

Analytical LCMS: purity 99.4% (System C, R_{T}=5.10 min), ES⁺: 267.4 [MH]⁺; HRMS calcd for C₁₅H₂₆N₂O₂: 266.1994, found 266.2002.

### Example 4

Piperidin-4-ylmethyl cyclopentylcarbamate hydrochloride

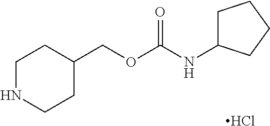

To a solution of tert-butyl 4-(hydroxymethyl)piperidine-1-carboxylate (Intermediate 1; 380 mg, 1.76 mmol) in anhydrous THF (5 mL) was added cyclopentyl isocyanate (0.220 mL, 1.95 mmol) and stirred over the weekend. The reaction mixture was concentrated in vacuo. The residue was dissolved in DCM (5 mL) and treated with TFA (2 mL). After stirring for 5 h the reaction mixture was concentrated in vacuo. The residue was dissolved in 1M Na₂CO₃ (50 mL) and extracted with EtOAc (3×50 mL). The combined organic phases were dried in vacuo and the residue purified by normal phase chromatography (gradient eluting with DCM/EtOH/aq NH3 mixture from ratio of 100:8:1 to 25:8:1). The product was dissolved in DCM (5 mL), treated with 2M HCl in Et₂O (1 mL, 2 mmol) and dried in vacuo to give piperidin-4-ylmethyl cyclopentylcarbamate hydrochloride (98 mg, 21%) as a white powder.

Analytical LCMS: purity 100% (System C, R_{T}=4.18 min), ES⁺: 226.9 [MH]⁺; HRMS calcd for C₁₂H₂₂N₂O₂: 226.1681, found 226.1681.

### Example 5

### [1-(2-Methoxyethyl)piperidin-4-yl]methyl cyclopentylcarbamate hydrochloride

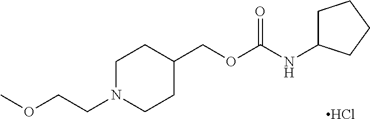

Piperidin-4-ylmethyl cyclopentylcarbamate (non HCl salt of Example 4; 323 mg, 1.43 mmol) was dissolved in DCM (5 mL) and treated with DIPEA (0.300 mL, 1.72 mmol) followed by (2-bromoethyl)methyl ether (0.135 mL, 1.44 mmol). After 48 hours the reaction mixture was poured onto 1M aq Na₂CO₃ solution (50 mL) and extracted with DCM (3×50 mL). The combined organic layers were dried (MgSO₄) and concentrated in is vacuo. The residue was purified by normal phase chromatography (gradient eluting with MeOH in EtOAc, from 0% to 30%) to give a colourless oil. This was dissolved in Et₂O (5 mL), treated with 2M HCl in Et₂O (0.5 mL) and concentrated in vacuo to give [1-(2-methoxyethyl)piperidin-4-yl]methyl cyclopentylcarbamate hydrochloride (77 mg, 17%) as a pale yellow glass.

Analytical LCMS: purity 100% (System C, R_{T}=4.98 min), ES⁺: 285.3 [MH]⁺; HRMS calcd for C₁₅H₂₈N₂O₃: 284.2100, found 284.2105.

### Example 6

### (1-Benzylpiperidin-4-yl)methyl cyclopentylcarbamate hydrochloride

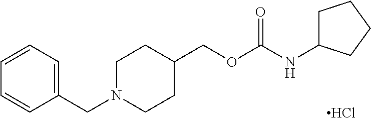

Piperidin-4-ylmethyl cyclopentylcarbamate (non HCl salt of Example 4; 445 mg, 1.97 mmol) was dissolved in DCM (5 mL), treated with benzyl bromide (0.24 mL, 2.0 mmol) and DIPEA (0.35 mL, 2.0 mmol) and stirred for 48 hours. The reaction mixture was poured onto 1M aq Na₂CO₃ solution (25 mL) and extracted with DCM (3×25 mL). The combined organic layers were dried (MgSO₄) and concentrated in vacuo to give a white solid (438 mg) which was purified by reverse phase chromatography followed by preparative HPLC. The residue was dissolved in Et₂O (5 mL), treated with 2M HCl in Et₂O (0.5 mL, 1 mmol)) and concentrated in vacuo to give (1-benzylpiperidin-4-yl)methyl cyclopentylcarbamate hydrochloride (157 mg, 23%) as a white solid.

Analytical HPLC: purity 100% (System B, R_{T}=4.80 min); Analytical LCMS: purity 98.7% (System C, R_{T}=5.67 min), ES⁺: 317.2 [MH]⁺; HRMS calcd for C₁₉H₂₈N₂O₂: 316.2151, found 316.2163.

### Example 7

### [1-(Cyanomethyl)piperidin-4-yl]methyl cyclopentylcarbamate hydrochloride

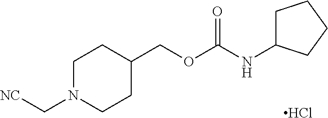

Piperidin-4-ylmethyl cyclopentylcarbamate (non HCl salt of Example 4; 458 mg, 2.0 is mmol) was dissolved in THF (10 mL), treated with DIPEA (0.350 mL, 2.0 mmol) and iodoacetonitrile (0.146 mL, 2.0 mmol) and stirred for 9 days. The reaction mixture was poured onto 1M aq Na₂CO₃ solution (100 mL) and extracted with DCM (3×100 mL). The combined organic layers were dried (MgSO₄) and concentrated in vacuo. The residue was dissolved in Et₂O (10 mL), treated with 2M HCl in Et₂O (1.5 mL, 3 mmol) and concentrated in vacuo to give a white solid. Recrystallisation from EtOAc/MeOH gave [1-(cyanomethyl)piperidin-4-yl]methyl cyclopentylcarbamate hydrochloride (201 mg, 33%) as a white solid.

Analytical LCMS: purity 100% (System C, R_{T}=6.30 min), ES⁺: 266.4 [MH]⁺. HRMS calcd for C₁₄H₂₃N₃O₂: 265.1790, found 265.1789.

### Example 8

### [1-(2-Methoxyethyl)piperidin-4-yl]methyl bicyclo[2.2.1]hept-2-ylcarbamate hydrochloride

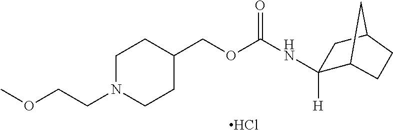

To a solution of (1-(2-methoxyethyl)piperidin-4-yl)methanol (Intermediate 3; 3.65 g, 21.1 mmol) and NMM (2.5 mL, 22.8 mmol) in DCM (100 mL) at 0° C. was added p-nitrophenyl chloroformate (4.42 g, 21.9 mmol). The reaction mixture was stirred at room temperature overnight and then washed with sat aq NaHCO₃ solution (5×100 mL), dried (MgSO₄) and concentrated in vacuo. The residue was recrystallised from EtOAc and heptane to give (1-(2-methoxyethyl)piperidin-4-yl)methyl 4-nitrophenyl carbonate (2.69 g) as an orange solid. The mother liquor was concentrated in vacuo and recrystallised from EtOAc to give a second crop (0.96 g, overall yield 51%).

Analytical LCMS: purity 100% (System C, R_{T}=1.59 min), ES⁺: 339.2 [MH]⁺.

To a solution of (1-(2-methoxyethyl)piperidin-4-yl)methyl 4-nitrophenyl carbonate (470 mg, 1.4 mmol) in DMF (15 mL) was added DIPEA (0.488 mL, 2.8 mmol), DMAP (10 mg, catalytic) and exo-2-aminonorbornane (0.248 ml, 2.1 mmol). The reaction mixture was stirred at room temperature for 18 hours and then concentrated in vacuo. The residue was dissolved in 1M aq HCl solution (10 mL) and washed with EtOAc (3×10 mL). The combined organic phases were dried (MgSO₄), filtered and concentrated in vacuo. The colourless oil obtained was purified by reverse phase chromatography (gradient eluting with MeOH in water, with 1% formic acid in each solvent). The colourless oil obtained was dissolved in MeOH (5 mL), 2M HCl in Et₂O (0.3 mL, 0.6 mmol) added and the solution concentrated in vacuo to give [1-(2-methoxyethyl)piperidin-4-yl]methyl bicyclo[2.2.1]hept-2-ylcarbamate hydrochloride (315 mg, 65%) as a white solid.

Analytical LCMS: purity 100% (System C, R_{T}=4.60 min), ES⁺: 311.1 [MH]⁺; HRMS calcd for C₁₇H₃₀N₂O₃: 310.2256, found mass: 310.2264.

### Biological Tests

Measurement of Overnight Body Weight Change in Male C57 bl/6 Mice

This model studies the effects of compounds on body weight gain during the pm-am period in order to maximise the effective window. Typically the mice gain about 1 g in weight during the dark phase and then loose the majority of this weight gain during the light phase, as represented in FIG. 1. The weight difference over any 24 hour period is very small whilst the weight difference between the beginning of the dark phase and the beginning of the light phase (pm-am) is maximal.

It is important to measure body weight change over the dark phase. If mice are dosed with an active compound on two consecutive days and the bodyweight change is recorded 48 hours after the first dose then no significant effect is observed. However if the body weight is change over the dark phase only is considered a significant and robust effect is seen. This is because the mice rebound during the light phase to compensate for the lack of weight gain over the dark phase. Very active long lasting compounds may also diminish this rebound and reduce the body weight over the 48 hours.

Weight Change Over Consecutive Days in C57bl\6 Male Mice:

The weight difference between the beginning of the dark phase and the beginning of the light phase (pm-am) is greater than the weight difference measured between pm and pm on 2 consecutive days. The effect of the compounds on the pm-am difference was therefore studied in order to maximise the effect window.

C57 bl/6 mice were grouped (5 per cage) and left 5 days for acclimatisation. A single intraperitoneally (ip) administered dose (60 mg/kg) was given just prior to the dark phase. Compounds were either water soluble or dissolved in up to 3% cremophor (in this case the vehicle also contained cremophor). The pH was adjusted from a minimum of 5.5 to a maximum of 8 depending on the nature of the compound.

As shown in FIGS. 2-3, compounds of Formula (I) are useful for decreasing body weight in mice.

### Leptin Assay in Non-Recombinant System

Although well-characterised in recombinant systems (e.g. ObRb-transfected HEK293 cells), where leptin elicits a very marked increase in STAT3 phosphorylation, these systems have often failed to provide an accurate measure of activity of a test compound towards the leptin receptor. It seems that overexpression of the receptor (as well as the possibility for different drugs to act on different parts of the signaling pathway triggered by leptin association with its receptor) results in most cases in the absence of activity of the drugs tested.

The leptin receptor expression in non-recombinant system is often fluctuating and care must be given to identify a system where signal stability remains within experiments. Using such a system, leptin receptor antagonist mimetics could be identified by evaluating is their action vs. leptin (see below).

Leptin is produced chiefly in adipose cells, but in humans, mRNA encoding leptin is also present in the placenta. Here, leptin might play an important proliferative role in the microvasculature. The possibility to use this hypothesis in a native cell line was evaluated.

### JEG-3 Protocol

In JEG-3 cells (choriocarcinoma cell line) leptin is able to stimulate proliferation up to 3 fold (Biol. Reprod. (2007) 76: 203-10). Leptin also causes a concentration-dependent increase in [³H]-thymidine incorporation in JEG-3 cells (FIG. 4, maximal effect at 100 nM (EC₅₀=2.1 nM)). The radioactivity incorporated by the cells is an index of their proliferative activity and is measured in counts per minute (CPM) with a liquid scintillation beta counter.

This finding can be applied to test whether a compound is able to either reproduce the effect of leptin on cell proliferation (leptin receptor agonist mimetic) (i.e., a given compound will cause an increase in incorporated [³H]-Thymidine by the cells) or to inhibit the effect of leptin (antagonistic effect) by preventing the leptin-mediated increase in [³H]-thymidine incorporation.

This approach has the advantage of using a non-recombinant system and has reasonable reproducibility and robustness.

### Measurement of Brain Penetration

The test species (rodent) is given a bolus dose of the substrate under investigation, usually via intravenous (IV) or oral (PO) routes. At appropriate time points, blood samples are taken and the resultant plasma extracted and analysed for substrate concentration and, where appropriate, metabolite concentration. At similar time points, animals from another group are sacrificed, brains isolated and the brain surface cleaned. Brain samples are then homogenised, extracted and analysed for substrate concentration and, where appropriate, metabolite concentration. Alternatively, microdialysis probes are implanted into one or more brain regions of the test species and samples collected at appropriate time points for is subsequent analysis. This method has the advantage of measuring only extra-cellular substrate concentration. Plasma and brain concentrations are then compared and ratios calculated, either by comparison of averaged concentrations at individual time points, or by calculation of the area-under-the-curve (AUC) of the concentration-time plots.

1. A compound of formula (I) 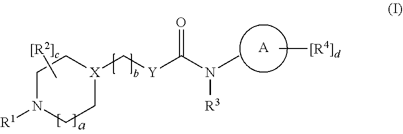
or a pharmaceutically acceptable salt, solvate, hydrate, geometrical isomer, tautomer, optical isomer or N-oxide thereof, wherein:
A is C₅₋₈-cycloalkyl;
X is N or C(H);
Y is O, N(R³) or CH₂;
R¹ is selected from hydrogen, C₁₋₆-alkyl, C₁₋₆-acyl (both optionally substituted with is one or more substituents independently selected from halogen, hydroxy, cyano and C₁₋₆-alkoxy), phenyl and benzyl (both optionally substituted with one or more substituents independently selected from halogen, hydroxy, cyano, nitro, CF₃, C₁₋₆-alkyl and C₁₋₆-alkoxy);
each R² is independently selected from halogen, hydroxy, C₁₋₆-alkyl and C₁₋₆-alkoxy (both optionally substituted with one or more substituents independently selected from halogen, hydroxy and C₁₋₆-alkoxy);
each R³ is independently selected from H and C₁₋₄-alkyl;
each R⁴ is independently selected from halogen, hydroxy, cyano, C₁₋₆-alkyl and C₁₋₆-alkoxy (both optionally substituted with one or more substituents independently selected from halogen, hydroxy and C₁₋₆-alkoxy), phenyl and benzyl (both optionally substituted with one or more substituents independently selected from halogen, hydroxy, cyano, nitro, CF₃, C₁₋₆-alkyl and C₁₋₆-alkoxy);
a is 0, 1 or 2;
b is 1 or 2; and
c and d are each independently 0, 1, 2 or 3;
provided that the compound is not selected from the group consisting of:
1-(3-chloro-2,2-dimethyl-1-oxopropyl)-N-[(cyclohexylamino)carbonyl]-4-piperidinemethanamine;
N-cyclohexyl-N′-[[1-[(4-methoxyphenyl)methyl]-4-piperidinyl]methyl]urea;
N-cyclohexyl-N,N′-dimethyl-N′-(4-piperidinylmethyl)urea;
4-piperidinylmethyl cyclohexylcarbamate;
2-[1-(phenylmethyl)-4-piperidinyl]ethyl cyclohexylcarbamate;
4-piperidinylmethyl cyclohexyl(methyl)carbamate;
2-(4-piperidinyl)ethyl cyclohexylcarbamate;
N-cyclopentyl-N,N′-dimethyl-N′-[2-(4-piperidinyl)ethyl]urea;
N′-cyclohexyl-N-methyl-N-(4-piperidinylmethyl)urea;
N-cyclohexyl-1-piperazinepropanamide;
N-(3-methylcyclohexyl)-1-piperazinepropanamide;
N-(2-methylcyclohexyl)-1-piperazinepropanamide;
N-(2,3-dimethylcyclohexyl)-1-piperazinepropanamide;
N-cyclohexyl-N′-[2-(1-piperazinyl)ethyl]urea;
2-(4-methylpiperazin-1-yl)ethyl (2-phenylbicyclo[2.2.1]hept-2-yl)carbamate;
N-cyclohexyl-1-piperazinebutanamide;
N-cyclohexyl-3-(1,4-diazepan-1-yl)propanamide;
3-(1,4-diazepan-1-yl)-N-(4-methylcyclohexyl)propanamide;
4-(4-chlorophenyl)-N-cyclohexyl-1-piperazinepropanamide;
4-(2-chlorophenyl)-N-cyclohexyl-N-methyl-1-piperazinepropanamide;
4-(3-chlorophenyl)-N-cyclohexyl-N-methyl-1-piperazinepropanamide;
4-(4-chlorophenyl)-N-cyclohexyl-N-methyl-1-piperazinepropanamide;
N-[2-[4-(2-methoxyphenyl)-1-piperazinyl]ethyl]-N′-(1-methylcyclohexyl)-urea;
N-cyclohexyl-4-(4-fluorophenyl)-N-methyl-1-piperazinepropanamide;
4-(2-methoxyphenyl)-N-(1-methylcyclohexyl)-1-piperazinepropanamide;
N-cyclohexyl-4-(4-methylphenyl)-1-piperazinepropanamide;
N-cyclo hexyl-4-(4-methoxyphenyl)-N-methyl-1-piperazinepropanamide;
N-cyclohexyl-N-methyl-4-(4-methylphenyl)-1-piperazinepropanamide;
N-cyclohexyl-4-(2-methoxyphenyl)-N-methyl-1-piperazinepropanamide;
N-cyclo hexyl-N-methyl-4-[3-(trifluoromethyl)phenyl]-1-piperazinepropanamide;
N-cyclopentyl-1-piperazinepropanamide;
N-cycloheptyl-1-piperazinepropanamide; and
N-cyclohexyl-N′-(4-piperidinylmethyl)urea.

2. A compound according to claim 1, wherein Y is O.

3. A compound according to claim 1 or 2, wherein X is C(H).

4. A compound according to any one of claims 1 to 3, wherein a is 1.

5. A compound according to any one of claims 1 to 4, wherein b is 1.

6. A compound according to claim 1, which is selected from:
(1-methylpiperidin-4-yl)methyl cyclopentylcarbamate;
(1-methylpiperidin-4-yl)methyl (1-phenylcyclopentyl)carbamate;
(1-methylpiperidin-4-yl)methyl bicyclo[2.2.1]hept-2-ylcarbamate;
piperidin-4-ylmethyl cyclopentylcarbamate;
[1-(2-methoxyethyl)piperidin-4-yl]methyl cyclopentylcarbamate;
(1-benzylpiperidin-4-yl)methyl cyclopentylcarbamate;
[1-(cyanomethyl)piperidin-4-yl]methyl cyclopentylcarbamate; and
[1-(2-methoxyethyl)piperidin-4-yl]methyl bicyclo [2.2.1]hept-2-ylcarbamate.

7. A pharmaceutical formulation containing a compound according to any one of claims 1 to 6 as active ingredient, in combination with a pharmaceutically acceptable diluent or carrier.

8. A compound according to any one of claims 1 to 6 for use in therapy.

9. A compound according to any one of claims 1 to 6 for use in the treatment or prevention of conditions or diseases associated with weight gain.

10. The compound according to claim 9, wherein the condition or disease is obesity, type 2 diabetes, lipodystrophy, insulin resistance, metabolic syndrome, hyperglycemia, hyperinsulinemia, dyslipidemia, hepatic steatosis, hyperphagia, hypertension, hypertriglyceridemia, infertility, a skin disorder associated with weight gain or macular degeneration.

11. A compound according to any one of claims 1 to 6 for use in the treatment or prevention of severe weight loss, dysmenorrhea, amenorrhea, female infertility or immunodeficiency, or in the treatment of wound healing.

12. A compound according to any one of claims 1 to 6 for use in the treatment or prevention of inflammatory conditions or diseases, low level inflammation associated with obesity and excess plasma leptin, atherosclerosis, macro or micro vascular complications of type 1 or 2 diabetes, retinopathy, nephropathy, autonomic neuropathy, or blood vessel damage caused by ischaemia or atherosclerosis.

13. A compound according to any one of claims 1 to 6 for use in the inhibition of angiogenesis.

14. Use of a compound according to any one of claims 1 to 6 in the manufacture of a medicament for the treatment or prevention of conditions or diseases associated with weight gain.

15. The use according to claim 14, wherein the condition or disease is obesity, type 2 diabetes, lipodystrophy, insulin resistance, metabolic syndrome, hyperglycemia, hyperinsulinemia, dyslipidemia, hepatic steatosis, hyperphagia, hypertension, hypertriglyceridemia, infertility, a skin disorder associated with weight gain or macular degeneration.

16. Use of a compound according to any one of claims 1 to 6 in the manufacture of a medicament for the treatment or prevention of severe weight loss, dysmenorrhea, amenorrhea, female infertility or immunodeficiency, or for the treatment of wound healing.

17. Use of a compound according to any one of claims 1 to 6 in the manufacture of a medicament for the treatment or prevention of inflammatory conditions or diseases, low level inflammation associated with obesity and excess plasma leptin, atherosclerosis, macro or micro vascular complications of type 1 or 2 diabetes, retinopathy, nephropathy, autonomic neuropathy, or blood vessel damage caused by ischaemia or atherosclerosis.

18. Use of a compound according to any one of claims 1 to 6 in the manufacture of a medicament for the inhibition of angiogenesis.

19. A method for treatment or prevention of conditions or diseases associated with weight gain, which comprises administering to a mammal, including man, in need of such treatment an effective amount of a compound according to any one of claims 1 to 6.

20. The method according to claim 19, wherein the condition or disease is obesity, type 2 diabetes, lipodystrophy, insulin resistance, metabolic syndrome, hyperglycemia, hyperinsulinemia, dyslipidemia, hepatic steatosis, hyperphagia, hypertension, hypertriglyceridemia, infertility, a skin disorder associated with weight gain or macular degeneration.

21. A method for treatment or prevention of severe weight loss, dysmenorrhea, amenorrhea, female infertility or immunodeficiency, or for treatment of wound healing, which comprises administering to a mammal, including man, in need of such treatment an effective amount of a compound according to any one of claims 1 to 6.

22. A method for treatment or prevention of inflammatory conditions or diseases, low level inflammation associated with obesity and excess plasma leptin, atherosclerosis, macro or micro vascular complications of type 1 or 2 diabetes, retinopathy, nephropathy, autonomic neuropathy, or blood vessel damage caused by ischaemia or atherosclerosis, which comprises administering to a mammal, including man, in need of such treatment an effective amount of a compound according to any one of claims 1 to 6.

23. A method for inhibition of angiogenesis, which comprises administering to a mammal, including man, in need of such treatment an effective amount of a compound according to any one of claims 1 to 6.
